(19) United States
(12) Patent Application Publication
(10) Pub. No.: US 2021/0146128 A1
(43) Pub. Date: May 20, 2021

(54) Face Lifting Device

(71) Applicants: Rose Pierre, West Hempstead, NY (US); Blandine Pierre, West Hempstead, NY (US)

(72) Inventors: Rose Pierre, West Hempstead, NY (US); Blandine Pierre, West Hempstead, NY (US)

(21) Appl. No.: 17/099,862

(22) Filed: Nov. 17, 2020

Related U.S. Application Data
(60) Provisional application No. 62/936,891, filed on Nov. 18, 2019.

(51) Int. Cl. A61N 1/32; A61N 1/36

(52) U.S. Cl. CPC A61N 1/328; A61N 1/36014

(57) ABSTRACT

A face lifting device is shown and described. The face lifting device is comprised of a facemask. The facemask has a plurality of electrodes secured to an interior side thereof. The electrodes are electrically coupled to a power source. The powersource is electrically coupled to an on/off device.

### CROSS REFERENCE TO RELATED APPLICATIONS

This application claims the benefit of U.S. Provisional Application No. 62/936,891 filed on Nov. 18, 2019. The above identified patent application is herein incorporated by reference in its entirety to provide continuity of disclosure.

### BACKGROUND OF THE INVENTION

The present invention relates to skin tightening and face lifting devices. More particularly, the present invention provides a mask which will work out face muscles to lift and tighten the face.

Many individuals wish to combat the effects of aging. In many cases this involves working on one's face. In some instances, the attempts to combat aging in the face include many different topical creams and oils. This can become monotonous and expensive over time. In many cases, there is also a significant trial an error process that is associated with picking a face regimen.

Further, in many instances these creams and oils only combat the aging effects of the skin. The current products on the market do not treat the underlying cause which would fix many aging effects in the face; the facial muscles. Using only topical treatments will not fix the entire problem and requires consistent maintenance.

Consequently, there is a need for an improvement in the art of face lifting. The present invention substantially diverges in design elements from the known art while at the same time solves a problem many people face when attempting to work on themselves or correct wrinkles or other perceived blemishes. In this regard the present invention substantially fulfills these needs.

### SUMMARY OF THE INVENTION

The present invention provides a face lifting device wherein the same can be utilized for providing convenience for the user when using a face lifting device. The face lifting device is comprised of a facemask. The facemask has a plurality of electrodes secured to an interior side thereof. The electrodes are electrically coupled to a power source. The power source is electrically coupled to an on/off device.

Another object of the face lifting device is to have the facemask electrically coupled to a control terminal. The control terminal houses the power source and the on/off device.

Another object of the face lifting device is to have a first adjustable headband secured to the facemask.

Another object of the face lifting device is to have a second adjustable headband secured to the facemask.

Another object of the face lifting device is to have a facemask which has a cutout in a location for eyes.

Another object of the face lifting device is to have a facemask which has a cutout for a mouth and nose.

Another object of the face lifting device is to have an adjustment knob secured to the facemask, wherein the adjustment knob electrically coupled between the power source and the electrodes.

Another object of the face lifting device is to have a gel located on the interior of the facemask, wherein the gel transfers electrical energy to the skin.

Another object of the face lifting device is to have a power source which is a rechargeable battery.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows front view of an embodiment of the face lifting device.

FIG. 2 shows a perspective view of an alternative embodiment of the face lifting device.

FIG. 3 shows rear view of an embodiment of the facemask for the face lifting device.

FIG. 4 shows a cross-sectional view of an embodiment of the facemask for the face lifting device.

FIG. 5 shows a wiring diagram of an embodiment of the face lifting device.

FIG. 6 shows a wiring diagram of an alternative embodiment of the face lifting device.

### LIST OF REFERENCE NUMERALS

With regard to the reference numerals used, the following numbering is used throughout the drawings.
101 Facemask
102 Eye cut out section
103 Nose/mouth cut out section
104 on/off switch
105 level adjustment knob
201 Control terminal
202 wire
301 Adjustable headband
302 Second adjustable headband
401 Gel layer
402 Top layer
403 Interior volume
404 Electrodes
501 Power source
502 Charging port
503 Power switch
504 Level adjuster
505 Electrical contacts
507 Muscles
601 Connection ports
101 AAA

### DETAILED DESCRIPTION OF THE INVENTION

Reference is made herein to the attached drawings. Like reference numerals are used throughout the drawings to depict like or similar elements of the face lifting device. For the purposes of presenting a brief and clear description of the present invention, a preferred embodiment will be discussed as used for the face lifting device. The figures are intended for representative purposes only and should not be considered to be limiting in any respect.

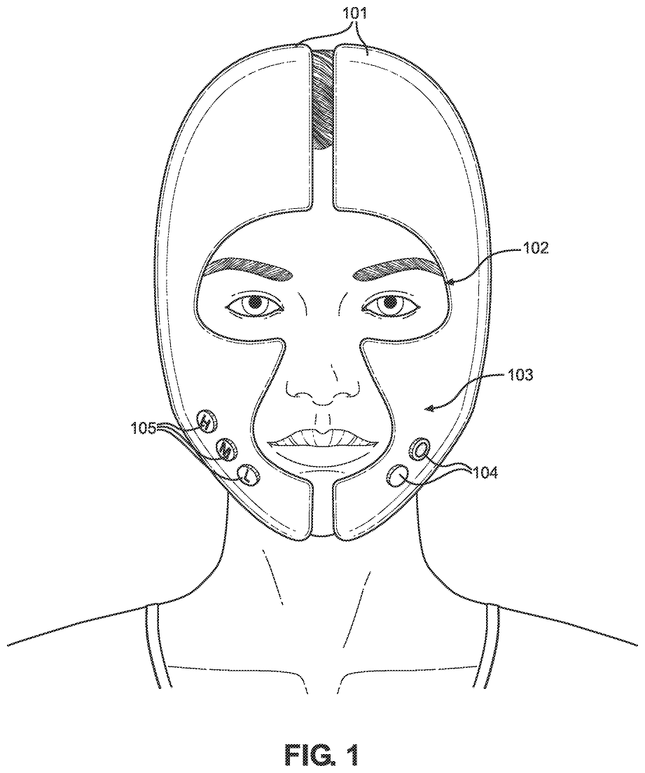

Referring now to FIG. 1, there is shown a front view of an embodiment of the face lifting device. The face lifting device will use electronic stimulation to tighten the facial muscles. The face lifting device is comprised of a facemask 101. The facemask 101 is configured to fit the contours of a human face. In some embodiments the facemask 101 is two sections held together as described in FIG. 3. In one embodiment, the facemask 101 has a section cut out 102 in the area of human eyes. In this embodiment the cut out 102 is located in a position where the human eye will be located when the facemask 101 is applied to the face. In one embodiment the human eye cut out 102 is oval shaped. This will ensure the eyes are not impeded by the facemask 101. In another embodiment, the facemask 101 has a section cut out 103 in the area of a human nose and mouth. In one embodiment the cutout 103 is a triangle shaped cut out. This will ensure that the human nose and mouth will fit within the same cutout. These sections 102, 103 will allow the facemask 101 to better fit to a face.

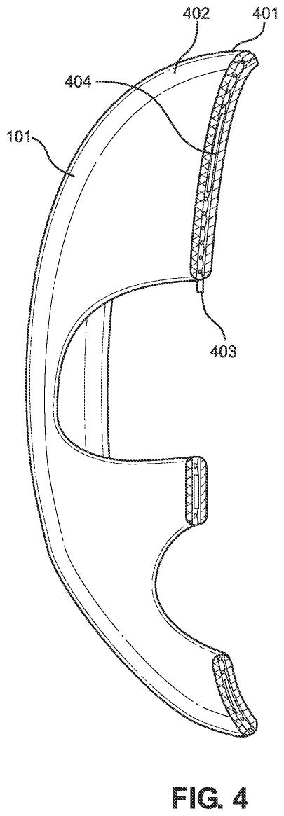

In one embodiment, the facemask 101 is a one size fits all facemask. This will be easy to manufacture. In another embodiment, the facemask 101 comes in different shapes and sizes. In yet another embodiment, the facemask 101 will be moderately customizable to fit to an individual face. This will be described the description of FIG. 4.

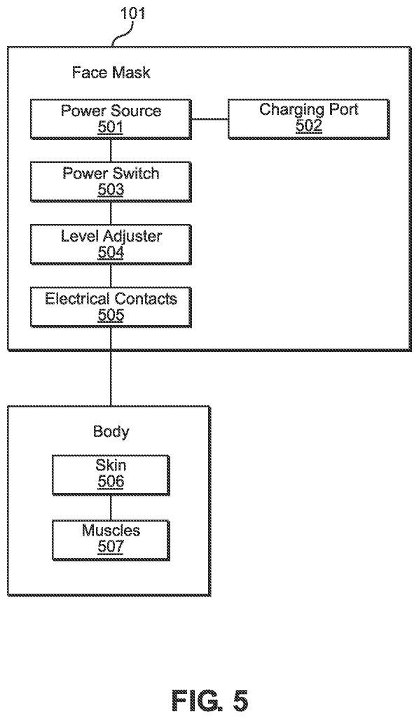

In one embodiment, the face lifting device is an all in one device. In this embodiment, the facemask 101 has an on and an off button 104 located thereon. In another embodiment a switch is used to turn the face listing device on and/or off. The on/off switch or on and off buttons 104 will activate the mask as described in FIG. 5. In one embodiment, the on and off buttons 104 have a different color or pattern to differentiate between the two. This will allow for easy operation and simple integration.

In one embodiment, the facemask 101 further includes a level adjustment knob. In another embodiment, as shown, the level adjustment is done using a plurality of buttons 105. The knob or buttons 105 will adjust the level of electric stimulation as described in FIG. 5. In one embodiment there are three buttons 105. In the shown embodiment each button 105 has an indicium thereon. The indicium will represent the level of the face lifting device.

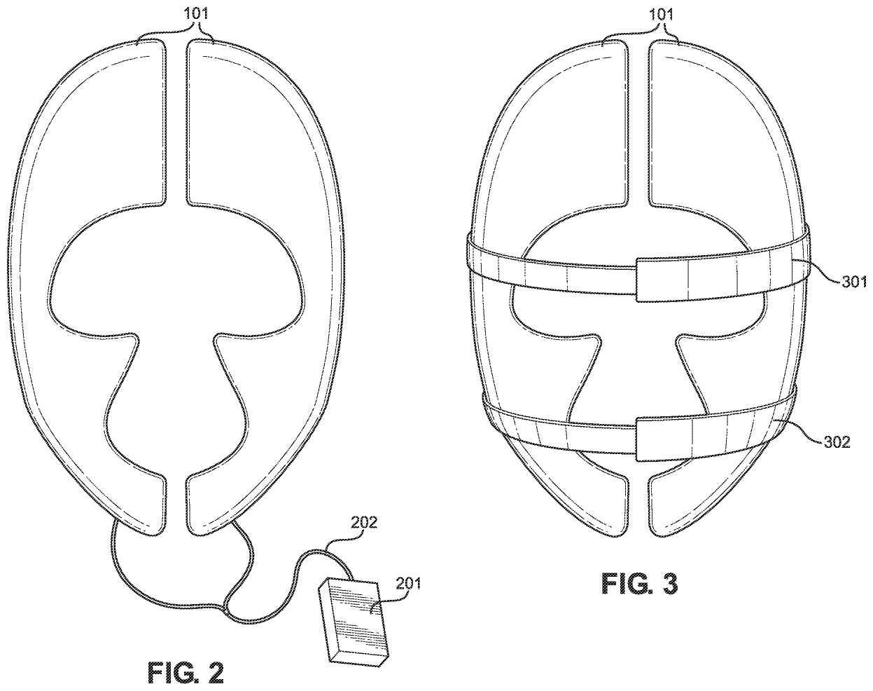

Referring now to FIG. 2, there is shown a perspective view of an alternative embodiment of the face lifting device. In another embodiment, the controls for the face lifting device are separate from the facemask 101. In this embodiment, there is a control terminal 201 connected to the facemask 101. In one embodiment, the control terminal 201 is connected to the facemask via a wire 202. In one embodiment, the facemask 101 is a multipart mask. In one embodiment, the wire 202 will split and connect to both sections of the facemask 101. In another embodiment, there will be two wires 202 that connect the control terminal 201 to the facemask 101.

Referring now to FIG. 3, there is shown rear view of an embodiment of the facemask for the face lifting device. In one embodiment, the facemask 101 has a first adjustable headband 301 secured thereto. In one embodiment, the first adjustable headband 301 is comprised of two parts slidably connected together. Each slidable part is connected to the facemask 101 at one end. In one embodiment, the first adjustable headband 301 is telescopically adjustable. This will allow the first adjustable headband 301 to support a one- or two-part facemask 101.

In one embodiment, the facemask 101 has a second adjustable headband 302 secured thereto. In one embodiment, the second adjustable headband 302 is comprised of two parts slidably connected together. Each slidable part is connected to the facemask 101 at one end. In one embodiment, the second adjustable headband 302 is telescopically adjustable. This will allow the second adjustable headband 302 to support a one- or two-part facemask 101.

In the embodiment with a first adjustable headband 301 and a second adjustable headband 302, the first adjustable headband 301 is secured to the facemask 101 at an area above the eye cutouts. The second adjustable headband 302 is secured to the facemask 101 in a lower cheek area. This will ensure that the facemask 101 is properly secured to the face.

Referring now to FIG. 4, there is shown a cross-sectional view of an embodiment of the facemask for the face lifting device. The facemask 101 is comprised of several layers. In one embodiment the facemask 101 has a gel layer 401. The gel layer 401 will rest upon the skin of an individual. The gel layer 401 is disposed along the interior side of the facemask 101. In one embodiment, the gel layer 401 will transmit electrical energy to the skin. In one embodiment the gel layer 401 is only a few millimeters thick. In one embodiment, the gel layer 401 is a thick layer. This will allow the gel layer 401 to function as a cushion. In one embodiment, the gel layer 401 will partially conform to a user's face. In this embodiment, the gel layer 401 will allow the facemask 101 to be partially customizable.

The facemask 101 has a top layer 402. In one embodiment the top layer 402 is made from a plastic material. Plastic is light weight and easy to clean. In another embodiment, the top layer 402 is comprised of a metal material. Metal is stiffer and will provide additional structure the facemask 101.

The top layer 402 and the gel layer 401 form an interior volume 403. The interior volume 403 will house various components of the face lifting device. In one embodiment, there are additional compartments formed within the top layer 402. This will allow for components to be placed within the facemask 101 as described herein.

In one embodiment, the interior volume 403 houses wires and electrodes 404. In one embodiment, the electrodes 404 will directly contact the skin. In another embodiment, the electrodes 404 will contact the gel layer 401.

Referring now to FIG. 5, there is shown a wiring diagram of an embodiment of the face lifting device. In one embodiment, the electrical components are all located within the facemask 101. The face lifting device is comprised of a power source 501. In one embodiment, the power source 501 is a battery. This will allow the power source 501 to be contained within the facemask 101. In one embodiment, the power source 501 is electrically coupled to a charging port 502. This will allow the power source 501 to be recharged if necessary. In another embodiment, the charging port 502 is replaced with a wireless charging device.

The power source 501 is electrically coupled to a power switch 503. The power switch 503 will allow power from the power source 501 to flow to the rest of the device. In one embodiment, the power switch 503 is electrically coupled to a power level adjuster 504. The power level adjuster 504 is comprised of a limiter that will control the power level. The power level adjuster 504 is electrically connected to electrical contacts 505. In another embodiment, the power switch 503 is coupled directly to the electrical contacts 505.

The electrical contacts 505 will transmit electrical energy to skin 506. As described in FIG. 4, the electrical contacts 505 will either directly contact the skin 506 or contact the skin 506 via the gel layer. Once the electrical energy is transferred to the skin 506, the electrical energy will stimulate the muscles 507. This will strengthen the muscles, thereby lifting the face.

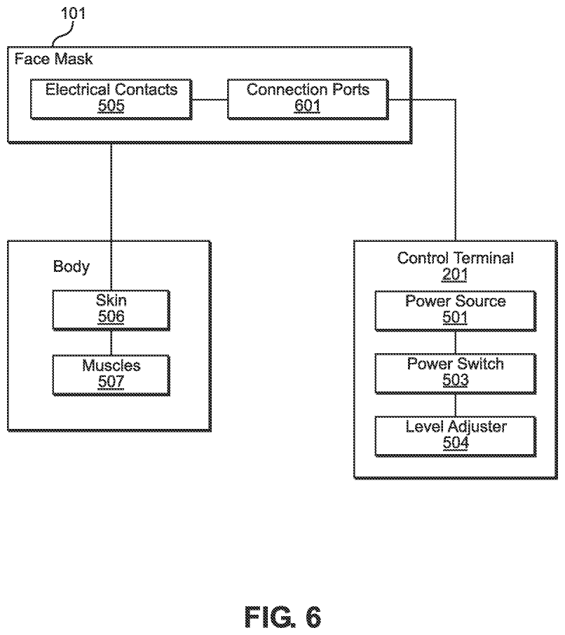

Referring now to FIG. 6, there is shown a wiring diagram of an alternative embodiment of the face lifting device. In another embodiment, the components are divided between the facemask 101 and a control terminal 201. This will allow for the facemask 101 to be easily disconnected from the control terminal 201. This embodiment will decrease the weight of the facemask 101, increasing comfort. In one embodiment, this will allow the facemask 101 to be disinfected. In another embodiment, this will allow the facemask 101 to be replaced without replacing the entire system.

The control terminal 201 will house the power source 501. In one embodiment, the power source 501 is a battery. In another embodiment, the power source 501 is directly from an outlet. The power source 501 is electrically coupled to a power switch 503. The power switch 503 will allow power from the power source 501 to flow to the rest of the device. In one embodiment, the power switch 503 is electrically coupled to a power level adjuster 504. The power level adjuster 504 is comprised of a limiter that will control the power level. The power level adjuster 504 is electrically connected to at least one connection port 601.

The connection port 601 is electrically coupled to electrical contacts 505. In another embodiment, the power switch 503 is coupled directly to the electrical contacts 505. The electrical contacts 505 will transmit electrical energy to skin 506. As described in FIG. 4, the electrical contacts 505 will either directly contact the skin 506 or contact the skin 506 via the gel layer. Once the electrical energy is transferred to the skin 506, the electrical energy will stimulate the muscles 507. This will strengthen the muscles lifting the face.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1) A face lifting device, the device comprising:
a facemask having an interior volume;
the facemask having a plurality of electrodes secured to an interior side thereof;
the electrodes are electrically coupled to a power source located within the facemask;
the power source is electrically coupled to an on/off device.

2) The face lifting device of claim 1, further comprising a first adjustable headband secured to the facemask.

3) The face lifting device of claim 2, further comprising a second adjustable headband secured to the facemask.

4) The face lifting device of claim 1, wherein the facemask has a cutout in a location for eyes.

5) The face lifting device of claim 1, wherein the facemask has a cutout for a mouth and nose.

6) The face lifting device of claim 1, wherein there is an adjustment knob secured to the facemask, wherein the adjustment knob is electrically coupled to the power source and the electrodes.

7) The face lifting device of claim 1, wherein there is a gel located on the interior of the facemask, wherein the gel transfers electrical energy to the skin.

8) The face lifting device of claim 1, wherein the power source is a rechargeable battery.

9) A face lifting device, the device comprising:
a facemask, wherein the facemask has a plurality of electrodes secured to an interior side thereof;
the electrodes are electrically coupled to a connection port on the facemask;
the connection port is electrically coupled to a wire;
the wire is electrically coupled to a control terminal.

10) The face lifting device of claim 9, further comprising a first adjustable headband secured to the facemask.

11) The face lifting device of claim 10, further comprising a second adjustable headband secured to the facemask.

12) The face lifting device of claim 9, wherein the facemask has a cutout in a location for eyes.

13) The face lifting device of claim 9, wherein the facemask has a cutout for a mouth and nose.

14) The face lifting device of claim 9, wherein the control terminal is electrically coupled to a power source.

15) The face lifting device of claim 9, further comprising an adjustment knob control terminal, wherein the adjustment knob is electrically coupled between the power source and the electrodes.

16) The face lifting device of claim 9, wherein there is a gel located on the interior of the facemask, wherein the gel transfers electrical energy to the skin.

17) The face lifting device of claim 9, wherein the power source is a rechargeable battery.

18) The face lifting device of claim 9, wherein there is an on/off switch electrically coupled to the control terminal.
